(19) United States
(12) Patent Application Publication
(10) Pub. No.: US 2021/0204872 A1
(43) Pub. Date: Jul. 8, 2021

(54) USE OF HEART RATE VARIABILITY (HRV) IN INFECTIOUS DISEASE PROGNOSIS AND TREATMENT

(71) Applicant: INMEDIX, INC., Normandy Park, WA (US)

(72) Inventor: Andrew James Holman, Seattle, WA (US)

(21) Appl. No.: 17/210,889

(22) Filed: Mar. 24, 2021

Related U.S. Application Data
(60) Provisional application No. 62/994,418, filed on Mar. 25, 2020.

(51) Int. Cl. A61B 5/00; A61B 5/024

(52) U.S. Cl. CPC A61B 5/4842; A61B 5/7275; A61B 5/02405

(57) ABSTRACT

Provided herein are methods of assessing prognosis for a subject having or suspected of having an infectious disease based on heart rate variability (HRV) of the subject. Also provided are methods of selecting an ANS optimizing therapy in a treatment for an infectious disease, based on HRV of the subject. Also provided are computer systems configured to perform aspects of the present methods.

### REFERENCE TO RELATED APPLICATIONS

This application claims priority to U.S. Provisional Application No. 62/994,418, filed Mar. 25, 2020, the entirety of which is incorporated herein by reference.

### BACKGROUND

### Field

Compositions and Methods in the field of medicine and infectious diseases are disclosed.

### Description of the Related Art

The immune system reacts to foreign threats, e.g. pathogens, cancer and foreign bodies. Its response can be gauged in two ways: sensitivity and virulence.

Sensitivity can have gradations due to the nature of the threat. Some pathogens can elicit an immediate response, while some do not. This may be to allow the body to respond to pathogens by generating immunologic alarm commensurate with their threat. Evolutionarily, slow immune responses can be detrimental. If a subject's immune system inappropriately responds slowly when intensive immune mobilization is desired, death may result. Survivors may tend to have greater inclination to respond quickly to that specific pathogen and propagate more.

Virulence is the intensity of immune activity—how aggressively the response is executed rather than how quickly the threat is appreciated.

Both components can be impacted by the brain through the autonomic nervous system (ANS), which in turn can be divided into the sympathetic (fight-or-flight) and parasympathetic (rest/restorative) components. Both components can be continuously active and competing for predominance depending on bodily needs—resting or fleeing. The former can act primarily through epinephrine and norepinephrine, while the latter can be managed primarily by acetylcholine.

### SUMMARY

Provided herein is a method of assessing prognosis for an infectious disease, comprising: measuring a heartbeat pattern of a subject having or suspected of having an infectious disease; calculating a heart rate variability (HRV) based on the measured heartbeat pattern, wherein the HRV indicates sympathetic activity levels, parasympathetic activity levels and/or an autonomic nervous system (ANS) balance of the subject; and assessing a prognosis for the infectious disease based on the HRV. Optionally, assessing the prognosis comprises assessing a prognosis for a treatment for the infectious disease.

In some embodiments, the treatment for the infectious disease comprises treatment of the subject in an intensive care unit (ICU) and/or using ventilator support.

In some embodiments, assessing the prognosis provides a favorable prognosis when the HRV indicates depressed sympathetic activity levels and/or elevated parasympathetic activity levels. In some embodiments, assessing the prognosis provides an unfavorable prognosis when the HRV indicates elevated sympathetic activity levels and/or depressed parasympathetic activity levels.

In some embodiments, the method further comprises treating the subject for the infectious disease based on the assessed prognosis. In some embodiments, the method includes treating the subject for the infectious disease upon an assessment providing a favorable prognosis. In some embodiments, the method includes treating the subject for the infectious disease upon an assessment providing an unfavorable prognosis. In some embodiments, the method includes withholding a treatment for the infectious disease from the subject upon an assessment providing a favorable prognosis. In some embodiments, the method includes withholding a treatment for the infectious disease from the subject upon an assessment providing an unfavorable prognosis.

In some embodiments, the method further comprises testing the subject for the infectious disease. Optionally, testing is performed before measuring the HRV.

In some embodiments, the method further comprising selecting an ANS optimizing therapy based on the HRV, wherein the selected ANS optimizing therapy improves the prognosis for a treatment for the infectious disease when the ANS optimizing therapy is co-administered to the subject with the treatment. Optionally, the ANS optimizing therapy comprises VNS and/or an ANS optimizing agent. In some embodiments, the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist. In some embodiments, the ANS optimizing agent comprises benzodiazepine or a selective serotonin reuptake inhibitor (SSRI). In some embodiments, the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone. In some embodiments, the ANS optimizing agent comprises one or more of lorazepam, clonazepam, pramipexole or trazodone.

In some embodiments, the method further comprises selecting a treatment for the infectious disease.

In some embodiments, the treatment comprises treatment of the subject in an intensive care unit (ICU), using a ventilator, and/or administration of a therapeutic agent to the subject.

Also provided herein is a method of selecting an ANS optimizing therapy in a treatment for an infectious disease, comprising: measuring a heartbeat pattern of a subject having an infectious disease; calculating a heart rate variability (HRV) based on the measured heartbeat pattern, wherein the HRV indicates sympathetic activity levels, parasympathetic activity levels and/or an autonomic nervous system (ANS) balance of the subject; and selecting an ANS optimizing therapy based on the HRV, wherein the selected ANS optimizing therapy improves the prognosis for a treatment of the infectious disease when the ANS optimizing therapy is co-administered to the subject with the treatment. In some embodiments, the method includes comprising co-administering to the subject: the treatment for the infectious disease; and an effective amount of the selected ANS optimizing therapy. In some embodiments, the method comprises withholding an ANS optimizing therapy based on the measured HRV.

In some embodiments, the treatment of the infectious disease comprises treatment of the subject in an intensive care unit (ICU) and/or using a ventilator. In some embodiments, the ANS optimizing therapy comprises vagus nerve stimulation (VNS) and/or an ANS optimizing agent. In some embodiments, the ANS optimizing therapy comprises VNS and/or an ANS optimizing agent.

In some embodiments, the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist. In some embodiments, the ANS optimizing agent comprises benzodiazepine or a selective serotonin reuptake inhibitor (SSRI). In some embodiments, the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone. In some embodiments, the ANS optimizing agent comprises one or more of lorazepam, clonazepam, pramipexole or trazodone.

In some embodiments, the treatment comprises treatment of the subject in an intensive care unit (ICU), using a ventilator, and/or administration of a therapeutic agent to the subject. In some embodiments, the therapeutic agent comprises an anti-infection agent, an immunosuppressant and/or an immunomodulator. In some embodiments, the immunomodulator comprises one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate. In some embodiments, the therapeutic agent comprises an anti-rheumatic drug. In some embodiments, the therapeutic agent comprises an IL-6 inhibitor. In some embodiments, the therapeutic agent comprises one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

In some embodiments, the anti-infection agent comprises one or more of an anti-viral agent, antibiotic, anti-fungal agent, and an anti-parasitic agent. In some embodiments, the anti-viral agent comprises one or more of acyclovir, famcyclovir, sorivudine, trifluorothymidine, valacyclovir, dideoxyinosine, interferon alpha, lamivudine, rifampicin, baloxavir marboxil, famiciclovir, letermovir, Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir, and Zidovudine. In some embodiments, the antibiotic comprises one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfasalazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin. In some embodiments, the anti-fungal agent comprises one or more of itraconazole, ketoconazole, fluoconazole, voriconazole, griseofulvin and amphotericin B.

In some embodiments, the infectious disease comprises a cold, pneumonia, or influenza.

In some embodiments, the infectious disease comprises a viral infection, fungal infection, a bacterial infection, and/or a parasitic infection. In some embodiments, the infectious disease comprises an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, and H1N1 virus.

In some embodiments, the infectious disease comprises a coronavirus infection. In some embodiments, the coronavirus infection comprises an infection by a Middle East respiratory syndrome coronavirus (MERS-CoV), severe acute respiratory syndrome coronavirus (SARS-CoV), or SARS-CoV-2 (COVID-19).

In some embodiments, the infectious disease comprises an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

In some embodiments, the infectious disease is associated with acute respiratory distress syndrome (ARDS) and/or a cytokine storm. In some embodiments, a targeted treatment for the infectious disease is not available. In some embodiments, a vaccine effective for the infectious disease is not available.

In some embodiments, the subject has acute respiratory distress syndrome (ARDS). In some embodiments, the subject has one or more comorbidities. Optionally, the one or more comorbidities comprise hypertension, diabetes and coronary heart disease.

In some embodiments, the subject does not have a comorbidity.

In some embodiments, measuring the heartbeat pattern comprises electrocardiography or pulse wave telemetry. In some embodiments, the HRV comprises Western standard HRV or Russian HRV. In some embodiments, the HRV comprises time-domain HRV, geometric domain HRV, and/or frequency-domain HRV. Optionally, the time-domain HRV comprises one or more of standard deviation between beats (SDNN), root mean square of successive differences (RMSSD), standard deviation of successive differences (SDSD), number of pairs of successive NNs that differ by more than 50 ms (NN50), proportion of NN50 divided by total number of NNs (pNN50), or estimated breath cycle (EBC).

In some embodiments, the prognosis comprises one or more of chance of survival, ventilator time, ICU time, hospitalization time, time to recovery, rehabilitation time, back-to-employment time, permanent disability, near-term and/or extended-term mortality.

Also provided herein is a computer system comprising a hardware processor and non-transitory memory comprising instructions stored thereon, wherein the instructions when executed by the hardware processor causes the processor to perform one or more aspects of the present methods. Optionally, the computer system comprises a tablet computer, a smartphone, a server or a desktop computer.

Also provided herein is a computer-readable medium comprising a software program that comprises code for performing or causing performing one or more aspects of the present methods.

In some embodiments, the method performed by the processor caused to be performed by the code further comprises selecting an ANS optimizing therapy based on the HRV, wherein the selected ANS optimizing therapy improves the prognosis for a treatment of the infectious disease when the ANS optimizing therapy is co-administered to the subject with the treatment. Optionally, the ANS optimizing therapy comprises VNS and/or an ANS optimizing agent.

### BRIEF DESCRIPTION OF THE DRAWINGS

FIG. 1 is a schematic diagram representing some methods of assessing prognosis for an infectious disease, according to embodiments of the present disclosure.

FIG. 2 is a schematic diagram representing some methods of selecting an ANS optimizing therapy in a treatment for an infectious disease, according to some embodiments of the present disclosure.

FIG. 3 is a schematic diagram showing a decision tree for selecting an ANS optimizing therapy in a treatment for an infectious disease, according to some embodiments of the present disclosure.

FIG. 4 is a schematic diagram representing a computer system configured to perform aspects of the present methods, according to some embodiments of the present disclosure.

### DETAILED DESCRIPTION

There are no targeted treatments yet for emerging infectious diseases, such as COVID-19. For example, 2-4% of COVID-19 infected patients can die from Acute Respiratory Distress Syndrome (ARDS) caused by an inflammatory cytokine storm. Currently, there are few insights as to why a specific patient develops cytokine storm, ARDS and death. Further, there are no effective options to address these and other severe symptoms of infectious diseases, such as COVID-19 infection, other than supportive care (e.g., 02, ventilator support, etc.).

### Methods

Provided herein are methods of assessing prognosis for an infectious disease, e.g., a viral disease (also referred to herein as “assessment methods”). In some embodiments, the assessment method comprises: measuring a heartbeat pattern in a subject having or suspected of having an infectious disease (e.g., by electrocardiography or pulse wave telemetry); calculating a heart rate variability (HRV) based on the measured heartbeat pattern, wherein the HRV indicates sympathetic activity levels, parasympathetic activity levels and/or an autonomic nervous system (ANS) balance of the subject; and assessing a prognosis for the infectious disease based on the HRV, e.g., the HRV measure of the subject's ANS state.

In some embodiments, one or more autonomous neuroregulatory therapies, e.g., ANS optimizing therapies, are administered to the subject based on the HRV, to improve the prognosis for the infectious disease. Without being bound by theory, ANS optimization is thought to reduce the risk of poor outcomes. In some embodiments, the ANS optimizing therapies include, without limitation, restless leg syndrome (RLS) drugs, autonomic neuroregulatory (e.g., ANS optimizing) drugs, and vagus nerve stimulation (e.g., via implantable or external devices). In some embodiments, poor ANS prognosis is defined by one of more of (i) high sympathetic and/or tension index, and low parasympathetic (e.g., Russian HRV), or (ii) low HF (“high frequency”) and/or total power, or (iii) high LF (“low frequency”)/HF ratio (1996 Western HRV).

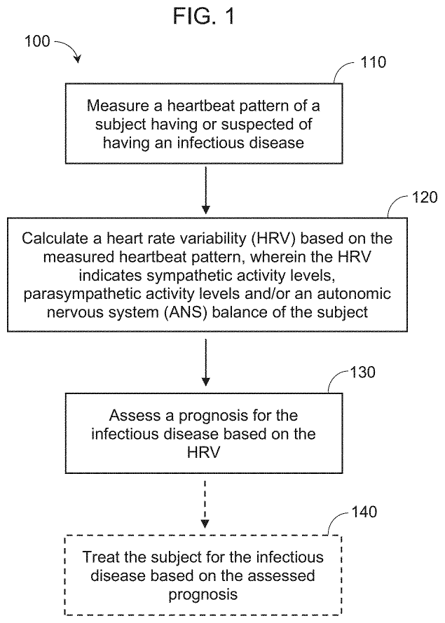

With reference to FIG. 1, some embodiments of some methods, e.g, assessment method, of the present disclosure is described. In some embodiments, the method 100 includes measuring 110 a heartbeat pattern of a subject having or suspected of having an infectious disease. The method can include calculating 120 heart rate variability (HRV) based on the measured heartbeat pattern, wherein the HRV indicates sympathetic activity levels, parasympathetic activity levels and/or an autonomic nervous system (ANS) balance of the subject. The method can further include assessing 130 a prognosis for the infectious disease based on the HRV. In some embodiments, the method includes treating 140 the subject for the infectious disease based on the assessed prognosis.

In some embodiments, a method of assessing prognosis for an infectious disease, comprises: obtaining a heartbeat pattern of a subject having or suspected of having an infectious disease; calculating a heart rate variability (HRV) based on the measured heartbeat pattern, wherein the HRV indicates sympathetic activity levels, parasympathetic activity levels and/or an autonomic nervous system (ANS) balance of the subject; and assessing a prognosis for the infectious disease based on the HRV, e.g., the HRV measure of the subject's ANS state. The heartbeat pattern of a subject can be obtained from any suitable source. In some embodiments, obtaining the heartbeat pattern of the subject includes measuring the heartbeat pattern of the subject, e.g., by electrocardiography or pulse wave telemetry. In some embodiments, obtaining the heartbeat pattern of the subject includes receiving data, e.g., electrocardiography or pulse wave telemetry data, containing the subject's heartbeat pattern from a remote source, e.g., a remote device or server.

Also provided herein are methods of selecting an ANS optimizing therapy in a treatment for an infectious disease (also referred to herein as “selection methods”). In some embodiments, the selection method includes measuring a heartbeat pattern of a subject having an infectious disease; calculating a heart rate variability (HRV) based on the measured heartbeat pattern, wherein the HRV indicates sympathetic activity levels, parasympathetic activity levels and/or an autonomic nervous system (ANS) balance of the subject; and selecting an ANS optimizing therapy based on the HRV, wherein the selected ANS optimizing therapy improves the prognosis for a treatment of the infectious disease when the ANS optimizing therapy is co-administered to the subject with the treatment.

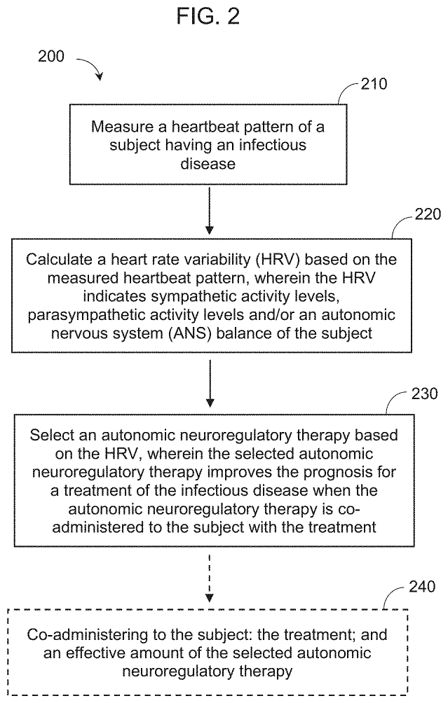

With reference to FIG. 2, an embodiment of a method, e.g., selection method, of the present disclosure is described. In some embodiments, the method 200 includes measuring 210 a heartbeat pattern of a subject having an infectious disease. The method can include calculating 220 heart rate variability (HRV) based on the measured heartbeat pattern, wherein the HRV indicates sympathetic activity levels, parasympathetic activity levels and/or an autonomic nervous system (ANS) balance of the subject. The method can further include selecting an ANS optimizing therapy based on the HRV, wherein the selected ANS optimizing therapy improves the prognosis for a treatment of the infectious disease when the ANS optimizing therapy is co-administered to the subject with the treatment. In some embodiments, the method includes co-administering to the subject: the treatment; and an effective amount of the selected ANS optimizing therapy.

The heartbeat pattern can be any suitable measure of heart activity (e.g., contraction) that is affected by the autonomic nervous system. In some embodiments, the heartbeat pattern is a measure of heart activity under autonomic control. In some embodiments, the heartbeat pattern is a measure of heart activity that is correlated with an activity level of one or more divisions of the autonomic nervous system (e.g., one or more of sympathetic activity level and parasympathetic activity level; an autonomic nervous system (ANS) balance). In some embodiments, the heartbeat pattern includes a temporal pattern of heart activity. The temporal pattern of heart contraction can include any suitable length of time over which to measure the heart activity. In some embodiments, the heartbeat pattern includes the heart activity over an interval of about 1, 2, 3, 4, 5, 6, 7, 8, 9, 10, 15, 20, 25, 30, 35, 40, 45, 50 60, 90, 120, 180 minutes, or any length of time within a range defined by any two of the preceding values. In some embodiments, the heartbeat pattern includes the heart activity over an interval of about 1, 2, 3, 4, 5, 6, 7, 8, 9, 10, 11, 12, 16, 20, 24, 28, 32, 36, 48, 60 hours, or any length of time within a range defined by any two of the preceding values. In some embodiments, the interval covered by the heartbeat pattern is a consecutive interval. In some embodiments, the interval covered by the heartbeat pattern does not include a substantial gap between successive beats of the heartbeat pattern.

The heartbeat pattern can be measured using any suitable option. In some embodiments, the heartbeat pattern is measured using electrocardiography or pulse wave telemetry. In some embodiments, the assessment method includes performing electrocardiography on the subject to measure the heartbeat pattern. Typically, the heartbeat pattern is measured for a sufficient time to allow for calculating the HRV. In some embodiments, the heartbeat pattern is measured for an interval of about 1, 2, 3, 4, 5, 6, 7, 8, 9, 10, 15, 20, 25, 30, 35, 40, 45, 50 60, 90, 120, 180 minutes, or any length of time within a range defined by any two of the preceding values. In some embodiments, the heartbeat pattern is measured for an interval of about 1, 2, 3, 4, 5, 6, 7, 8, 9, 10, 11, 12, 16, 20, 24, 28, 32, 36, 48, 60 hours, or any length of time within a range defined by any two of the preceding values. In some embodiments, the interval over which the heartbeat pattern is measured is a substantially continuous interval. In some embodiments, the interval over which the heartbeat pattern is measured does not include a substantial gap between successive beats of the heartbeat pattern.

Calculating the HRV can include calculating any suitable measure of HRV based on the measured heartbeat pattern. Generally, the HRV indicates sympathetic activity levels, parasympathetic activity levels and/or an autonomic nervous system (ANS) balance of the subject. Without being bound by theory, inspiration reduces chest cavity pressure to facilitate inhalation. That reduction in pressure can slightly expand the aorta. Baroreceptors within the wall of the aorta can register this as a transient reduction in blood pressure and can alert the brain—the ANS—to increase heart rate through the parasympathetic vagus nerve. With exhalation, the inverse can occur. Thus, breathing can result in slight accelerations and decelerations in heart rate, and such changes in heart rate may be manifested as heart rate variability (HRV). This phenomenon can be abolished by increasing sympathetic tone, and hence ANS state can be gauged through, in some embodiments, calculating the standard deviation between beats (SDNN) to obtain an example of time-domain HRV.

In some embodiments, the HRV is Western standard HRV, Russian HRV, or any other suitable HRV measure, including, without limitation, non-FDA approved HRV measures available (e.g., for use in sports and wellness applications). In some embodiments, the HRV includes time-domain HRV, geometric domain HRV, and/or frequency-domain HRV. In some embodiments, the HRV is time-domain HRV. Any suitable time-domain HRV may be calculated based on the measured heartbeat pattern. Suitable time-domain HRV includes, without limitation, standard deviation between beats (SDNN), root mean square of successive differences (RMSSD), standard deviation of successive differences (SDSD), number of pairs of successive NNs that differ by more than 50 ms (NN50), proportion of NN50 divided by total number of NNs (pNN50), and estimated breath cycle (EBC).

In some embodiments, the HRV is frequency-domain HRV. Without being bound by theory, breathing can be most frequent and the high frequency (HF) output of the frequency-domain HRV can almost exclusively be reflective of parasympathetic activity. Low frequency (LF) oscillation in beat-to-beat changes by frequency-domain analysis can reflect a combination of sympathetic and parasympathetic activity. In some embodiments, the frequency-domain HRV is the ratio: LF/HF, reflecting ANS balance. In some embodiments, the HF output includes a frequency in a range of about 0.1 to 1 Hz, e.g., about 0.1 to 0.8 Hz, about 0.1 to 0.6 Hz, about 0.15 to 0.5 Hz, including about 0.2 to 0.4 Hz. In some embodiments, the LF output includes a frequency in a range of about 0.01 to 0.2 Hz, about 0.02 to 0.2 Hz, about 0.03 to 0.15 Hz, including about 0.04 to about 0.15 Hz. Any suitable option may be used to calculate the frequency-domain HRV. In some embodiments, calculating frequency-domain HRV includes taking the Fast Fourier transform of the measured heartbeat pattern. In some embodiments, the frequency-domain HRV is calculated based on a heartbeat pattern containing a 5-minute beat-to-beat rhythm strip.

In some embodiments, the HRV is informed by geometric-domain analysis. In some embodiments, the geometric-domain HRV provides a first output that correlates with a sympathetic activity level, and a second output that is Bayevsky's tension index.

In some embodiments, the HRV indicates depressed sympathetic activity levels and/or elevated parasympathetic activity levels. In some embodiments, the HRV provides a favorable or unfavorable prognosis. In some embodiments, assessing the prognosis provides a favorable prognosis when the HRV indicates depressed sympathetic activity levels and/or elevated parasympathetic activity levels. In some embodiments, a calculated HRV that is greater than a threshold HRV indicates depressed sympathetic activity levels and/or elevated parasympathetic activity levels. In some embodiments, a calculated HRV that is greater than a threshold HRV indicates a favorable prognosis. In some embodiments, the HRV indicates elevated sympathetic activity levels and/or depressed parasympathetic activity levels. In some embodiments, assessing the prognosis provides an unfavorable prognosis when the HRV indicates elevated sympathetic activity levels and/or depressed parasympathetic activity levels. In some embodiments, a calculated HRV that is less than a threshold HRV indicates elevated sympathetic activity levels and/or depressed parasympathetic activity levels. In some embodiments, a calculated HRV that is less than a threshold HRV provides an unfavorable prognosis. In some embodiments, the HRV is represented as a continuous variable.

In some embodiments, the threshold values described herein are set by population percentages. For example, in some embodiments, a population of people is examined for the variable (e.g., HRV or other variable) and the resulting values determined across the population are divided into quartiles, with the top 25% percent being the most extreme level, the next 25% being moderate high, the next 25% being moderate low, and the final 25% being lowest for of the variable for the population. These values can then be used to establish cutoff values for particular actions for any single patient.

The HRV can be calculated using any suitable option. In some embodiments, the HRV is calculated using a computer system configured to receive data containing the measured heartbeat pattern. In some embodiments, the computer system includes a processor configured and non-transitory memory containing instructions, which when executed by the processor causes the processor to perform at least a portion of the present assessment methods. In some embodiments, the processor performs measuring the heartbeat pattern of the subject. In some embodiments, the processor performs calculating the HRV based on the measured heartbeat pattern of the subject. In some embodiments, the processor performs assessing a prognosis for the infectious disease based on the HRV. In some embodiments, the processor performs recommending a treatment for the infectious disease based on the prognosis. In some embodiments, the computer system is a tablet computer, smartphone, a server (e.g., Microsoft Azure or other cloud service) or a desktop computer.

In some embodiments, the method includes assessing a prognosis for the infectious disease based on the HRV. In some embodiments, the method includes assessing a prognosis for the infectious disease based on the HRV and one or more factors indicative of the subject's ANS profile. In some embodiments, the factors indicative of the subject's ANS profile are clinical factors. In some embodiments, one or more of the subject's clinical history, genetic makeup and life history are factors indicative of the subject's ANS profile.

As used herein, “prognosis” has its ordinary and customary meaning, in view of the present disclosure. “Prognosis” can refer to one or more potential outcomes for a condition or disease of a subject (e.g., a patient) at one or more points or periods of time in the future. The likelihood of the outcome in a prognosis may vary depending on the embodiment. In some embodiments, the prognosis is a likely outcome. In some embodiments, the prognosis is a reasonably certain outcome. In some embodiments, the likelihood of the outcome is 10% or more, e.g., 20% or more, 30% or more, 40% or more, 50% or more, 60% or more, 70% or more, 80% or more, 90% or more, 95% or more, including about 100%, or any percentage within a range defined by any two of the preceding values. In some embodiments, the prognosis includes one or more of chance of survival, ventilator time, intensive care unit (ICU) time, hospitalization time, time to recovery, rehabilitation time, back-to-employment time, permanent disability, near-term and/or extended-term mortality rate.

In some embodiments, the likelihood of an outcome is expressed as an odds ratio. In some embodiments, selection of the treatment for the infectious disease and/or an ANS optimizing therapy is based on the odds ratio for one or more outcomes. In some embodiments, an odds ratio determined based on the calculated HRV indicates a favorable prognosis. In some embodiments, an odds ratio determined based on the calculated HRV indicates an unfavorable prognosis.

In some embodiments, the prognosis includes prognosis for a treatment for the infectious disease. In some embodiments, assessing prognosis includes assessing a prognosis for a treatment for the infectious disease. In some embodiments, the treatment for the infectious disease comprises treatment of the subject in an intensive care unit (ICU) and/or using ventilator support. The prognosis can include an outcome for one or more of a variety of treatments of the infectious disease, as described herein. Thus, those patients with a high likelihood of the disorder can be assigned such critical medical care equipment earlier, while those with a low likelihood need not receive such critical medical care. Such information can also be used to distribute medical equipment within the population or a hospital for example. Thus, in some embodiments, the prognosis of multiple patients can be used, in combination, to distribute device within a facility. This can also alter how much medical equipment may need to be purchased at any given time, by a care facility.

In some embodiments, assessing the prognosis provides a favorable prognosis when the HRV indicates depressed sympathetic activity levels and/or elevated parasympathetic activity levels. In some embodiments, a favorable prognosis includes an improved outcome for the subject compared to an average patient with the infectious disease. In some embodiments, a favorable prognosis includes an improved outcome for the subject compared to an average patient with the infectious disease and having the same clinical profile as the subject. In some embodiments, a favorable prognosis includes an improved outcome for the subject compared to an average patient with the infectious disease and having the same comorbidities as the subject. In some embodiments, a favorable prognosis includes an improved outcome compared to a current state of the subject. In some embodiments, a favorable prognosis includes complete or partial recovery from the infectious disease without the need for medical intervention, e.g., without the need for hospitalization. In some embodiments, a favorable prognosis includes complete or partial recovery from the infectious disease without the need for resource-intensive treatment for the infectious disease, e.g., without the need for treatment in an intensive care unit (ICU) and/or using ventilator support. In some embodiments, a favorable prognosis includes an improved outcome after the subject receives a treatment for the infectious disease, e.g., in a hospital, in an intensive care unit (ICU), and/or using ventilator support.

In some embodiments, a depressed sympathetic activity levels is one that is lower than 50% of the population. In some embodiments, it is one that is in the lower 25% of the population or even lower 10% or even lower 5% of a population. In some embodiments, an elevated parasympathetic activity is one that is in the upper 50% of the population, e.g., upper 25%, upper 10%, or even upper 5% of a population. In some embodiments, the population is defined as a geographical population. In some embodiments, the population is one of at least 10 or more similarly defined subjects (e.g., same sex, age, and/or general fitness, etc.) In some embodiments, the population is at least 10, 20, 50, 100, 1000, or 10,000 or more subjects.

In some embodiments, a favorable prognosis includes a reduced risk of suffering a cytokine storm due to the infectious disease. In some embodiments, an unfavorable prognosis includes an elevated risk of suffering a cytokine storm due to the infectious disease. In some embodiments, a favorable prognosis includes a reduced risk of suffering from ARDS due to the infectious disease. In some embodiments, an unfavorable prognosis includes an elevated risk of suffering from ARDS due to the infectious disease.

In some embodiments, a favorable prognosis includes an improved chance of survival compared to a reference percentage (e.g., an average chance of survival for a patient with the infectious disease, an average chance of survival for a patient with the infectious disease and having the same clinical profile as the subject, an average chance of survival for a patient with the infectious disease and having the same comorbidities as the subject, etc.). In some embodiments, a favorable prognosis includes a generally desirably high percentage for chance of survival. In some embodiments, a favorable prognosis includes a reduced near-term mortality compared to a reference near-term mortality (e.g., an average near-term mortality for a patient with the infectious disease, an average near-term mortality for a patient with the infectious disease and having the same clinical profile as the subject, an average near-term mortality for a patient with the infectious disease and having the same comorbidities as the subject, etc.). In some embodiments, a favorable prognosis includes a generally desirably low near-term mortality. In some embodiments, a favorable prognosis includes a reduced extended-term mortality compared to a reference extended-term mortality (e.g., an average extended-term mortality for a patient with the infectious disease, an average extended-term mortality for a patient with the infectious disease and having the same clinical profile as the subject, an average extended-term mortality for a patient with the infectious disease and having the same comorbidities as the subject, etc.). In some embodiments, a favorable prognosis includes a generally desirably low extended-term mortality. In some embodiments, a favorable prognosis includes a reduced risk of permanent disability compared to a reference risk of permanent disability (e.g., an average risk of permanent disability for a patient with the infectious disease, an average risk of permanent disability for a patient with the infectious disease and having the same clinical profile as the subject, an average risk of permanent disability for a patient with the infectious disease and having the same comorbidities as the subject, etc.). In some embodiments, a favorable prognosis includes a generally desirably low risk of permanent disability. In some embodiments, a favorable prognosis includes reduced ventilator time (e.g., time during which ventilator support is required) compared to a reference ventilator time (e.g., an average ventilator time for a patient with the infectious disease, an average ventilator time for a patient with the infectious disease and having the same clinical profile as the subject, an average ventilator time for a patient with the infectious disease and having the same comorbidities as the subject, etc.). In some embodiments, a favorable prognosis includes a generally desirably short ventilator time. In some embodiments, a favorable prognosis includes reduced ICU time (e.g., time spent in the ICU) compared to a reference ICU time (e.g., an average ICU time for a patient with the infectious disease, an average ICU time for a patient with the infectious disease and having the same clinical profile as the subject, an average ICU time for a patient with the infectious disease and having the same comorbidities as the subject, etc.). In some embodiments, a favorable prognosis includes a generally desirably short ICU time. In some embodiments, a favorable prognosis includes reduced hospitalization time (e.g., time during which hospital care is required) compared to a reference hospitalization time (e.g., an average hospitalization time for a patient with the infectious disease, an average hospitalization time for a patient with the infectious disease and having the same clinical profile as the subject, an average hospitalization time for a patient with the infectious disease and having the same comorbidities as the subject, etc.). In some embodiments, a favorable prognosis includes a generally desirably short hospitalization time. In some embodiments, a favorable prognosis includes reduced time to recovery compared to a reference time to recovery (e.g., an average time to recovery for a patient with the infectious disease, an average time to recovery for a patient with the infectious disease and having the same clinical profile as the subject, an average time to recovery for a patient with the infectious disease and having the same comorbidities as the subject, etc.). In some embodiments, a favorable prognosis includes a generally desirably short time to recovery for a patient with the infectious disease. In some embodiments, a favorable prognosis includes reduced rehabilitation time compared to a reference rehabilitation time (e.g., an average rehabilitation time for a patient treated for the infectious disease, an average rehabilitation time for a patient treated for the infectious disease and having the same clinical profile as the subject, an average rehabilitation time for a patient treated for the infectious disease and having the same comorbidities as the subject, etc.). In some embodiments, a favorable prognosis includes a generally desirably short rehabilitation time for a patient with the infectious disease. In some embodiments, a favorable prognosis includes reduced back-to-employment time (e.g., time from when the subject becomes unable to continue in his/her regular course of employment because the infectious disease until the subject regains his/her employment status) compared to a reference back-to-employment time (e.g., an average back-to-employment time for a patient with the infectious disease, an average back-to-employment time for a patient with the infectious disease and having the same clinical profile as the subject, an average back-to-employment time for a patient with the infectious disease and having the same comorbidities as the subject, etc.). In some embodiments, a favorable prognosis includes a generally desirably short back-to-employment time for a patient with the infectious disease.

In some embodiments, a favorable prognosis indicates an outcome improved by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference outcome. For example, in some embodiments, a favorable prognosis includes a chance of survival that is increased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference percentage. For example, in some embodiments, a favorable prognosis includes a ventilator time reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%, or by a percentage within a range defined by any two of the preceding values, compared to a reference ventilator time. In some embodiments, a favorable prognosis indicates an outcome improved by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to the subject before receiving treatment for the infectious disease.

In some embodiments, assessing the prognosis provides an unfavorable prognosis when the HRV indicates elevated sympathetic activity levels and/or depressed parasympathetic activity levels. In some embodiments, an unfavorable prognosis includes a worse outcome for the subject compared to an average patient with the infectious disease. In some embodiments, an unfavorable prognosis includes a worse outcome for the subject compared to an average patient with the infectious disease and having the same clinical profile as the subject. In some embodiments, an unfavorable prognosis includes a worse outcome for the subject compared to an average patient with the infectious disease and having the same comorbidities as the subject. In some embodiments, an unfavorable prognosis includes a worse outcome compared to a current state of the subject. In some embodiments, an unfavorable prognosis includes a need for medical intervention, e.g., need for hospitalization, to prevent death or permanent disability due to the infectious disease, and/or to achieve at least some recovery from the infectious disease. In some embodiments, an unfavorable prognosis includes a need for resource-intensive treatment for the infectious disease, e.g., treatment in an intensive care unit (ICU) and/or using ventilator support, to prevent death or permanent disability due to the infectious disease, and/or to achieve at least some recovery from the infectious disease. In some embodiments, an unfavorable prognosis includes disease progression after the subject receives a treatment for the infectious disease, e.g., in a hospital, in an intensive care unit (ICU), and/or using ventilator support.

In some embodiments, an unfavorable prognosis includes a worse chance of survival compared to a reference percentage (e.g., an average chance of survival for a patient with the infectious disease, an average chance of survival for a patient with the infectious disease and having the same clinical profile as the subject, an average chance of survival for a patient with the infectious disease and having the same comorbidities as the subject, etc.). In some embodiments, an unfavorable prognosis includes a generally undesirably low percentage for chance of survival. In some embodiments, an unfavorable prognosis includes an increased near-term mortality compared to a reference near-term mortality (e.g., an average near-term mortality for a patient with the infectious disease, an average near-term mortality for a patient with the infectious disease and having the same clinical profile as the subject, an average near-term mortality for a patient with the infectious disease and having the same comorbidities as the subject, etc.). In some embodiments, an unfavorable prognosis includes a generally undesirably high near-term mortality. In some embodiments, an unfavorable prognosis includes an increased extended-term mortality compared to a reference extended-term mortality (e.g., an average extended-term mortality for a patient with the infectious disease, an average extended-term mortality for a patient with the infectious disease and having the same clinical profile as the subject, an average extended-term mortality for a patient with the infectious disease and having the same comorbidities as the subject, etc.). In some embodiments, an unfavorable prognosis includes a generally undesirably high extended-term mortality. In some embodiments, an unfavorable prognosis includes an increased risk of permanent disability compared to a reference risk of permanent disability (e.g., an average risk of permanent disability for a patient with the infectious disease, an average risk of permanent disability for a patient with the infectious disease and having the same clinical profile as the subject, an average risk of permanent disability for a patient with the infectious disease and having the same comorbidities as the subject, etc.). In some embodiments, an unfavorable prognosis includes a generally undesirably high risk of permanent disability. In some embodiments, an unfavorable prognosis includes increased ventilator time (e.g., time during which ventilator support is required) compared to a reference ventilator time (e.g., an average ventilator time for a patient with the infectious disease, an average ventilator time for a patient with the infectious disease and having the same clinical profile as the subject, an average ventilator time for a patient with the infectious disease and having the same comorbidities as the subject, etc.). In some embodiments, an unfavorable prognosis includes a generally long ventilator time. In some embodiments, an unfavorable prognosis includes increased ICU time (e.g., time spent in the ICU) compared to a reference ICU time (e.g., an average ICU time for a patient with the infectious disease, an average ICU time for a patient with the infectious disease and having the same clinical profile as the subject, an average ICU time for a patient with the infectious disease and having the same comorbidities as the subject, etc.). In some embodiments, an unfavorable prognosis includes a generally long ICU time. In some embodiments, an unfavorable prognosis includes increased hospitalization time (e.g., time during which hospital care is required) compared to a reference hospitalization time (e.g., an average hospitalization time for a patient with the infectious disease, an average hospitalization time for a patient with the infectious disease and having the same clinical profile as the subject, an average hospitalization time for a patient with the infectious disease and having the same comorbidities as the subject, etc.). In some embodiments, an unfavorable prognosis includes a generally long hospitalization time. In some embodiments, an unfavorable prognosis includes increased time to recovery compared to a reference time to recovery (e.g., an average time to recovery for a patient with the infectious disease, an average time to recovery for a patient with the infectious disease and having the same clinical profile as the subject, an average time to recovery for a patient with the infectious disease and having the same comorbidities as the subject, etc.). In some embodiments, an unfavorable prognosis includes a generally long time to recovery for a patient with the infectious disease. In some embodiments, an unfavorable prognosis includes increased rehabilitation time compared to a reference rehabilitation time (e.g., an average rehabilitation time for a patient treated for the infectious disease, an average rehabilitation time for a patient treated for the infectious disease and having the same clinical profile as the subject, an average rehabilitation time for a patient treated for the infectious disease and having the same comorbidities as the subject, etc.). In some embodiments, an unfavorable prognosis includes a generally long rehabilitation time for a patient with the infectious disease. In some embodiments, an unfavorable prognosis includes increased back-to-employment time (e.g., time from when the subject becomes unable to continue in his/her regular course of employment because the infectious disease until the subject regains his/her employment status) compared to a reference back-to-employment time (e.g., an average back-to-employment time for a patient with the infectious disease, an average back-to-employment time for a patient with the infectious disease and having the same clinical profile as the subject, an average back-to-employment time for a patient with the infectious disease and having the same comorbidities as the subject, etc.). In some embodiments, an unfavorable prognosis includes a generally long back-to-employment time for a patient with the infectious disease.

In some embodiments, an unfavorable prognosis indicates an outcome worsened by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference outcome. For example, in some embodiments, an unfavorable prognosis includes a chance of survival that is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to a reference percentage. For example, in some embodiments, an unfavorable prognosis includes a ventilator time increased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, about 100%, or by a percentage within a range defined by any two of the preceding values, compared to a reference ventilator time. In some embodiments, an unfavorable prognosis indicates an outcome worsened by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100% or more, or by a percentage within a range defined by any two of the preceding values, compared to the subject before receiving treatment for the infectious disease.

In some embodiments, the method includes treating the subject for the infectious disease based on the assessed prognosis. Without being bound by theory, a favorable ANS profile (either as a categorical or continuous variable of HRV) may be predictive of a less virulent infection and better outcome not requiring ANS optimization. In some embodiments, patients with a favorable ANS profile (high parasympathetic and low sympathetic activity) are treated with standard of infectious care. If upon screening (e.g., by measuring the heartbeat pattern and calculating the HRV), a patient having a favorable ANS profile can, in some embodiments, return home to convalesce, while another patient having the same level of symptomatology with poor ANS profile can be treated more aggressive, e.g., greater monitoring, quick hospitalization, faster advancement to more aggressive antibiotics (antivirals, etc.). In some embodiments, e.g., when hospital resources are limited and demand exceeds supply, a patient having a more favorable ANS profile may be treated over another patient having a less favorable ANS profile to maximize the chances of successfully treating at least one patient.

In some embodiments, the method includes treating the subject for the infectious disease upon an assessment providing a favorable prognosis. In some embodiments, the method includes treating the subject for the infectious disease upon an assessment providing an unfavorable prognosis. In some embodiments, treating a subject having an unfavorable prognosis can include providing more intensive treatment of the infectious disease (e.g., administration of stronger medication, use of ICU and/or ventilator, etc.). In some embodiments, the method includes withholding a treatment for the infectious disease from the subject upon an assessment providing a favorable prognosis. In some embodiments, the method includes withholding a treatment for the infectious disease from the subject upon an assessment providing an unfavorable prognosis. In some embodiments, resource for treatment of a subject is limited, and the method includes withholding a treatment for the infectious disease from the subject upon an assessment providing an unfavorable prognosis. In some embodiments, resource for treatment of a subject is limited when there are not enough medical equipment (e.g., ICU equipment, ventilators, etc.) for all subjects in need of treatment by such medical equipment to receive the treatment.

The treatment can include any suitable treatment for the infectious disease. In some embodiments, the subject is treated in an intensive care unit (ICU) and/or using ventilator support. In some embodiments, the treatment includes administering one or more therapeutic agents to the subject, as described herein.

In some embodiments, the method includes testing the subject for the infectious disease. The subject may be tested for the infectious disease at any suitable time. In some embodiments, testing for the infectious disease is performed before, after, or concurrently to measuring the HRV. Testing for the infectious disease can be performed using any suitable option. In some embodiments, testing includes a clinical observation and/or diagnostic testing.

In some embodiments, a method of the present disclosure, e.g., assessment methods, includes selecting an ANS optimizing therapy based on the HRV, wherein the selected ANS optimizing therapy improves the prognosis for the subject having the infectious disease. In some embodiments, a method of the present disclosure, e.g., selection methods, includes selecting an ANS optimizing therapy based on the HRV, wherein the selected autonomic optimizing neuroregulatory therapy improves the prognosis for a treatment of the infectious disease when the ANS optimizing therapy is co-administered to the subject with the treatment for the infectious disease. In some embodiments, an ANS optimizing therapy is selected based on one or more additional factors. The additional factors may include any suitable factor that can affect the prognosis. In some embodiments, the additional factors include, without limitation, age, presence or absence of comorbidities, gender, clinical history, life history, etc.

In some embodiments, the ANS optimizing therapy includes vagus nerve stimulation (VNS) and/or an ANS optimizing agent. VNS may be performed using any suitable option, e.g., to optimize the subject's ANS. In some embodiments, the VNS is delivered using an external or implantable device. In some embodiments, the VNS is delivered using a non-invasive technique. In some embodiments, an implantable VNS device delivers stimulation along any suitable position along the vagus nerve. Suitable VNS options include, without limitation, those provided by SetPoint Medical and Galvani Bioelectronics.

Without being bound by theory, it is thought the main ANS parasympathetic conduit, the vagus nerve, can impact immune function. Afferent vagal signals can alert the brain to inflammation, while efferent signals can reduce inflammation through activation of α-7 nicotinic acetylcholine receptors in the spleen, thereby reducing levels of proinflammatory cytokines, IL-1, IL-1 and TNF to reduce inflammation.

Vagus nerve stimulation can reduce mouse inflammation to increased survival in a bacterial pathogen model (compared to controls). For example, transection of the vagus accelerated death in the same model. Thus, vagus nerve stimulation (VNS) can reduce inflammation in various conditions and diseases.

In some embodiments, the therapeutic agent is an anti-infection agent, an immunosuppressant and/or an immunomodulatory, as described herein.

The ANS optimizing therapy may be selected based on any suitable measure of the HRV, as described herein. In some embodiments, the selected ANS optimizing therapy reduces sympathetic activity level of the subject. In some embodiments, the selected ANS optimizing therapy increases parasympathetic activity level of the subject. In some embodiments, the selected ANS optimizing therapy restores ANS balance in the subject. In some embodiments, an ANS optimizing therapy that reduces sympathetic activity level and/or increases parasympathetic activity level of the subject is selected upon calculating an HRV that indicates an unfavorable prognosis. In some embodiments, an ANS optimizing therapy that reduces sympathetic activity level, increases parasympathetic activity level, and/or restores ANS balance of the subject is selected upon calculating an HRV that indicates an unfavorable prognosis, as described herein. In some embodiments, an ANS optimizing therapy that reduces sympathetic activity level, increases parasympathetic activity level, and/or restores ANS balance of the subject is selected upon calculating an HRV that indicates an unfavorable prognosis for a treatment of the infectious disease.

In some embodiments, the method includes co-administering to the subject: an effective amount of the therapeutic agent; and an effective amount of the selected ANS optimizing therapy, thereby treating the infectious disease. In some embodiments, the method includes withholding an ANS optimizing therapy based on the measured HRV. In some embodiments, the therapeutic agent is administered to the subject before, after, or concurrently to administering the selected ANS optimizing therapy. In some embodiments, the therapeutic agent and the selected ANS optimizing therapy are co-administered when one is administered within a time interval in which the other retains its physiological effect on the subject.

An “effective amount” or “therapeutically effective amount” can be a quantity of a therapeutic agent or ANS optimizing agent sufficient to achieve a desired effect in a subject being treated. For instance, this can be the amount necessary to prevent, reduce or inhibit one or more signs or symptoms associated with an infectious disease.

The therapeutic agent can be administered using any suitable method, and may vary depending on the therapeutic agent. In some embodiments, the therapeutic agent is administered orally, parenterally, or topically. In some embodiments, the therapeutic agent is administered intravenously, intra-arterially, subcutaneously, intraperitoneally, intramuscularly, etc.

In some embodiments, the method, e.g., the selection method, includes recommending the ANS optimizing therapy selected for the subject. In some embodiments, the method includes recommending a therapeutic agent to be co-administered with the ANS optimizing therapy selected for the subject. In some embodiments, the method includes recommending to withhold, avoid, or terminate an ANS optimizing therapy based on the measured HRV. Recommending can be done using any suitable option. In some embodiments, where the selecting is performed on a computer system, e.g., a tablet computer, the recommending can include displaying the recommendation on a screen on the computer system. In some embodiments, where the recommending can include sending a communication, e.g., e-mail, fax, text message, etc., that includes the recommendation.

Depending on the embodiment, a variety of ANS optimizing agents can be used in methods of the present disclosure. In some embodiments, the ANS optimizing agent includes, without limitation, one or more of an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist. In some embodiments, the ANS optimizing agent includes, without limitation, one or more of benzodiazepine or a selective serotonin reuptake inhibitor (SSRI). In some embodiments, the ANS optimizing agent includes, without limitation, one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone. In some embodiments, the ANS optimizing agent includes, without limitation, one or more of lorazepam, clonazepam, pramipexole or trazodone.

Depending on the embodiment, a variety of therapeutic agents can be used in methods of the present disclosure. In some embodiments, the therapeutic agent includes, without limitation, one or more of an anti-infection agent, an immunosuppressant and/or an immunomodulatory. In some embodiments, the immunomodulator includes, without limitation, one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

In some embodiments, the therapeutic agent includes an anti-rheumatic drug. In some embodiments, the therapeutic agent includes an IL-6 inhibitor. In some embodiments, the therapeutic agent includes, without limitation, one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

In some embodiments, the anti-infection agent includes, without limitation, one or more of an anti-viral agent, antibiotic, anti-fungal agent, and an anti-parasitic agent. In some embodiments, the anti-viral agent includes, without limitation, one or more of acyclovir, famcyclovir, ganciclovir, foscarnet, idoxuridine, sorivudine, trifluorothymidine, valacyclovir, vidarabine, didanosine, dideoxyinosine, stavudine, zalcitabine, zidovudine, amantadine, interferon alpha, ribavirin and rimantadine. In some embodiments, the antibiotic includes, without limitation, one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfasalazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin. In some embodiments, the anti-fungal agent include, without limitation, one or more of itraconazole, ketoconazole, fluoconazole, voriconazole, griseofulvin and amphotericin B.

The disclosed agents and compounds may be used alone or in combination with other treatments. These agents or compounds, when used in combination with other agents, may be administered as a daily dose or an appropriate fraction of the daily dose (e.g., bid). The compounds may be administered after a course of treatment by another agent, during a course of therapy with another agent, administered as part of a therapeutic regimen, or may be administered prior to therapy by another agent in a treatment program.

Examples of pharmaceutically acceptable salts include acetate, adipate, besylate, bromide, camsylate, chloride, citrate, edisylate, estolate, fumarate, gluceptate, gluconate, glucuronate, hippurate, hyclate, hydrobromide, hydrochloride, iodide, isethionate, lactate, lactobionate, maleate, mesylate, methylbromide, methylsulfate, napsylate, nitrate, oleate, palmoate, phosphate, polygalacturonate, stearate, succinate, sulfate, sulfosalicylate, tannate, tartrate, terphthalate, tosylate, and triethiodide.

Compositions containing the active ingredient may be in any form suitable for the intended method of administration. Suitable routes of administration include, for example, oral, rectal, topical, nasal, pulmonary, ocular, intestinal, and parenteral administration. Suitable routes of parenteral administration include, without limitation, intravenous, intramuscular, and subcutaneous administration. Other suitable routes of administration include, without limitation, intraperitoneal, intra-arterial, intra-articular, intracardiac, intracisternal, intradermal, intralesional, intraocular, intrapleural, intrathecal, intrauterine, and intraventricular administration. The infectious disease to be treated, along with the physical, chemical, and biological properties of the agent being administered, can dictate the type of formulation and the route of administration to be used, as well as whether local or systemic delivery would be preferred.

When the compounds are administered via oral administration, for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient can be mixed with an inert solid diluent, for example calcium phosphate or kaolin, or as soft gelatin capsules wherein the active ingredient can be mixed with water or an oil medium, such as peanut oil, liquid paraffin or olive oil.

Formulations suitable for parenteral administration include aqueous and non-aqueous isotonic sterile injection solutions which may contain, for example, antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

In some embodiments unit dosage formulations contain a daily dose or unit, daily sub-dose, or an appropriate fraction thereof, of a drug. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

The present embodiments include pharmaceutical formulations comprising one or more compounds described throughout in association with a pharmaceutically acceptable carrier. Preferably these formulations are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual, buccal, topical or rectal administration, or for administration by inhalation or insufflation. Also, the instant compounds can be administered to the body through Xenoport technology. XenoPort identifies and characterizes transporters throughout the body that are useful to drug delivery, then uses selected transporter proteins as “targets” and employs medicinal chemistry techniques to modify drugs into substrates for these transporters.

Alternatively, the formulations may be presented in a form suitable for once-daily, once-weekly or once-monthly administration; for example, an insoluble salt of the active compound may be adapted to provide a preparation for intramuscular injection. The pharmaceutical formulations described herein can be administered to a patient per se, or in pharmaceutical formulations where they are mixed with other active ingredients, as in combination therapy, or suitable pharmaceutically acceptable carriers or excipient(s). Techniques for formulation and administration of the compounds of the instant application may be found in “Remington's Pharmaceutical Sciences,” Mack Publishing Co., Easton, Pa., 18th edition, 1990.

For preparing solid pharmaceutical formulations such as tablets, the principal active ingredient is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation formulation containing a homogeneous mixture of a compound of the present embodiments, or a pharmaceutically acceptable salt thereof. When referring to these preformulation formulations as homogeneous, it is meant that the active ingredient is dispersed evenly throughout the formulation so that the formulation may be readily subdivided into equally effective unit dosage forms such as tablets, pills and capsules. This solid preformulation formulation is then subdivided into unit dosage forms of the type described above containing from about 0.01 to about 10,000 mg of the compounds of the present embodiments. Preferably the dosage is from about 50 to about 5000 mg; more preferably, the dosage is from about 450 to about 1800 mg; even more preferably, the dosage is from about 600 to about 1000 mg. The tablets or pills of the novel formulation can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Furthermore, compounds for the present embodiments can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

Pharmaceutical formulations for parenteral administration, e.g., by bolus injection or continuous infusion, include aqueous solutions of the active compounds in water-soluble form. Additionally, suspensions of the active compounds may be prepared as appropriate oily injection suspensions. Suitable lipophilic solvents or vehicles include fatty oils such as sesame oil, or other organic oils such as soybean, grapefruit or almond oils, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For oral administration, the instant compounds can be formulated readily by combining the active compounds with pharmaceutically acceptable carriers well known in the art. Such pharmaceutically acceptable carriers enable the compounds of the present embodiments to be formulated as tablets, pills, dragees, capsules, liquids, gels, syrups, slurries, suspensions and the like, for oral ingestion by a patient to be treated. Pharmaceutical formulations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

For buccal administration, the pharmaceutical formulations may take the form of tablets, lozenges, wafers and rapid-dissolve preparations formulated in conventional manner.

The compounds of the present embodiments can also be administered in the form of liposome pharmaceutical formulations, such as small unilamellar vesicles, large unilamellar vesicles and multilamellar vesicles. Liposomes can be formed from a variety of phospholipids, such as cholesterol, stearylamine or phosphatidylcholines.

Further disclosed herein are various pharmaceutical formulations well known in the pharmaceutical art for uses that include intraocular, intranasal, and intraauricular delivery. Suitable penetrants for these uses are generally known in the art. Pharmaceutical formulations for intraocular delivery include aqueous ophthalmic solutions of the active compounds in water-soluble form, such as eyedrops, or in gellan gum (Shedden et al., Clin. Ther., 23(3):440-50 (2001)) or hydrogels (Mayer et al., Ophthalmologica, 210(2):101-3 (1996)); ophthalmic ointments; ophthalmic suspensions, such as microparticulates, drug-containing small polymeric particles that are suspended in a liquid carrier medium (Joshi, A., J. Ocul. Pharmacol., 10(1):29-45 (1994)), lipid-soluble formulations (Alm et al., Prog. Clin. Biol. Res., 312:447-58 (1989)), and microspheres (Mordenti, Toxicol. Sci., 52(1):101-6 (1999)); and ocular inserts.

Alternatively, other delivery systems for hydrophobic pharmaceutical compounds may be employed. Liposomes and emulsions are well known examples of delivery vehicles or pharmaceutical acceptable carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The dosage regimen utilizing the compounds of the present embodiments is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the pharmaceutical formulation's availability to target sites. This involves a consideration of the distribution, equilibrium, and elimination of the compounds. Advantageously, compounds of the present embodiments may be administered, for example, in a single daily dose, or the total daily dosage may be administered in divided doses of two, three or four times daily.

In the methods of the present embodiments, the pharmaceutical formulations herein described in detail are typically administered in accordance with conventional pharmaceutical practices.

For instance, for oral administration in the form of a tablet or capsule, the compounds of the present embodiments can be combined with an oral, non-toxic pharmaceutically acceptable inert carrier such as ethanol, glycerol, water and the like. Moreover, when desired or necessary, suitable pharmaceutically acceptable carriers, such as, binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Some examples of pharmaceutically acceptable carriers or diluents for therapeutic use are well known in the pharmaceutical art, and are described, for example, in Remington's Pharmaceutical Sciences, 18th Ed., Mack Publishing Co., Easton, Pa. (1990), which is incorporated herein by reference in its entirety.

The oral liquid formulations in which the present embodiments may be incorporated for administration orally include using pharmaceutically acceptable carriers, aqueous solutions, suitably flavoured syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous oral suspensions include synthetic and natural gums such as tragacanth, acacia, alginate, dextran, sodium carboxymethylcellulose, methylcellulose, polyvinyl-pyrrolidone or gelatin. Other dispersing agents which may be employed include glycerin and the like.

The daily dosage of the products may be varied over a wide range; e.g., from about 10 to about 10,000 mg per adult human per day. For oral administration, the formulations are preferably provided in the form of tablets containing about 0.001, 0.01, 0.1, 1, 10.0, 15.0, 25.0, 50.0, 100, 200, 300, 400, 500, 600, 700, 800, 900 1000, 2000, 3000, 4000, 5000, 6000, 7000, 8000, 9000 or 10,000 milligrams of the active ingredient for the symptomatic adjustment of the dosage to the patient to be treated. The instant pharmaceutical formulations typically contain from 10 mg to about 2000 mg of the instant compounds, preferably, from about 50 mg to about 1000 mg of active ingredient. An effective amount of the instant compounds is ordinarily supplied at a dosage level of from about 0.002 mg/kg to about 150 mg/kg of body weight per day. Preferably, the range is from about 0.02 to about 80 mg/kg of body weight per day, and especially from about 0.2 mg/kg to about 40 mg/kg of body weight per day. The compounds may be administered on a regimen of about 1 to about 10 times per day.

The subject in the various methods of the present disclosure can be any suitable subject having or suspected of having an infectious disease. In some embodiments, the subject is a mammal (e.g., mouse, rat, dog, cat, horse, pig, cow, camel, rabbit, sheep, primate, including non-human primate, or human). In some embodiments, the subject has acute respiratory distress syndrome (ARDS). In some embodiments, the subject has developed ARDS as a consequence of having the infectious disease. In some embodiments, the subject has one or more comorbidities. The subject may have a variety of comorbidities, depending on the embodiment. In some embodiments, the subject has one or more of hypertension, diabetes and coronary heart disease. In some embodiments, the subject does not have a comorbidity.

In some embodiments, the subject is diagnosed with an infectious disease. In some embodiments, the subject exhibits one or more symptoms of an infectious disease. The subject may exhibit a variety of symptoms depending on the infectious disease. In some embodiments, the subject exhibits one or more of a cough, dry cough, headache, joint pain, muscle ache, nausea, dizziness, fever, lethargy, sore throat, nasal drip, etc.

According to some embodiments, the subject has or is suspected of having one or more of a variety of infectious diseases. In some embodiments, the infectious disease includes, without limitation, a cold, pneumonia, or influenza. In some embodiments, the infectious disease is associated with acute respiratory distress syndrome (ARDS) and/or a cytokine storm. In some embodiments, a subject having the infectious disease has a chance of developing ARDS and/or a cytokine storm of about 1% or more, 2% or more, 3% or more, 4% or more, 5% or more, 10% or more, 15% or more, 20% or more, including 25% or more, or a percentage within a range defined by any two of the preceding values. In some embodiments, a subject having the infectious disease and one or more comorbidities (e.g., high blood pressure, diabetes, coronary heart disease, etc.) has a chance of developing ARDS and/or a cytokine storm of about 1% or more, 2% or more, 3% or more, 4% or more, 5% or more, 10% or more, 15% or more, 20% or more, including 25% or more, or a percentage within a range defined by any two of the preceding values. In some embodiments, the infectious disease increases the chances of the subject having the disease developing acute respiratory distress syndrome (ARDS) and/or a cytokine storm.

In some embodiments, a targeted treatment for the infectious disease is not available. In some embodiments, a targeted treatment for the infectious disease is not available when there is no therapeutic agent known or available to specifically target the infectious agent (e.g., virus, bacteria, fungus, parasite, etc.) to treat the infectious disease. In some embodiments, a targeted treatment for the infectious disease is not available when there is no vaccine effective against the infectious agent causing the infectious disease is available. In some embodiments, a targeted treatment for the infectious disease is not available when there is no antibiotic, anti-viral, anti-fungal, or anti-parasitic agent effective against the infectious agent causing the infectious disease is available.

In some embodiments, the infectious disease includes, without limitation, a viral infection, fungal infection, a bacterial infection, and/or a parasitic infection. In some embodiments, the infectious disease includes, without limitation, an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, human immunodeficiency virus (HIV), and H1N1 virus.

In some embodiments, the infectious disease the infectious disease is a coronavirus infection. In some embodiments, the coronavirus infection includes an infection by, without limitation, a Middle East respiratory syndrome coronavirus (MERS-CoV), severe acute respiratory syndrome coronavirus (SARS-CoV), or SARS-CoV-2 (COVID-19).

In some embodiments, the infectious disease includes, without limitation, an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

### Systems and Softwares

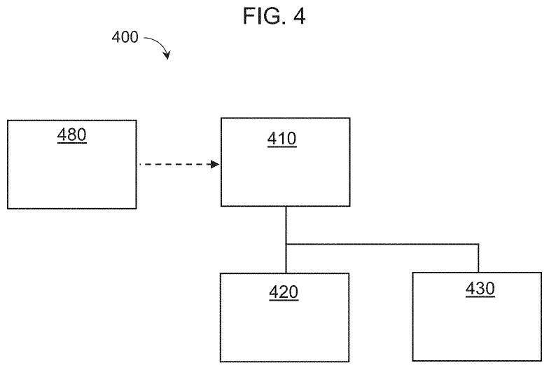

Also provided herein is a computer system comprising a hardware processor and non-transitory memory comprising instructions stored thereon, wherein the instructions when executed by the hardware processor causes the processor to perform one or more aspects of the methods (e.g., assessment methods, selection methods) of the present disclosure. With reference to FIG. 4, an embodiment of a computer system of the present disclosure is described. The computer system 400 can include a hardware processor 410 and a non-transitory memory 420 storing instructions, where the instructions when executed by the hardware processor causes the processor to perform one or more aspects of the present methods (e.g., assessment methods, selection methods). In some embodiments, the computer system includes display 430. In some embodiments, the computer system includes one or more input devices (e.g., keyboard, mouse, touchscreen, microphone, camera, video camera, etc.). In some embodiments, the computer system is configured to receive heartbeat pattern data from a device 480 (e.g., an electrocardiography system, a pulse wave telemetry system) configured to measure the heartbeat pattern of a subject having or suspected of having an infectious disease. In some embodiments, the computer system includes a device 480 (e.g., an electrocardiography system, a pulse wave telemetry system) configured to measure the heartbeat pattern of a subject having or suspected of having an infectious disease.

In some embodiments, the computer system is configured to perform one or more of measuring the heartbeat pattern, and calculating the HRV of the assessment methods, as described herein. In some embodiments, the computer system is configured to perform one or more of obtaining the heartbeat pattern, and calculating the HRV of the assessment methods, as described herein. In some embodiments, the computer system includes a non-transitory memory that includes data associating a calculated HRV with a prognosis for the infectious disease and/or a recommended ANS optimizing therapy and/or therapeutic agent that will provide for a favorable prognosis, as described herein. In some embodiments, the computer system is, without limitation, a tablet computer, a smartphone, a server or a desktop computer. In some embodiments, the computer system is a cloud computing platform, such as, but not limited to, Microsoft Azure, Amazon Web Services, Google Cloud, Oracle Cloud, IBM Cloud, Alibaba Cloud, etc.

Also provided herein is a computer-readable medium comprising a software program that comprises code for performing or causing performing one or more features of the methods (e.g., assessment methods, selection methods) of the present disclosure. In some embodiments, the software program includes code for performing one or more of measuring the heartbeat pattern, and calculating the HRV of the assessment methods, as described herein. In some embodiments, the software program includes code for performing one or more of obtaining the heartbeat pattern, and calculating the HRV of the assessment methods, as described herein.

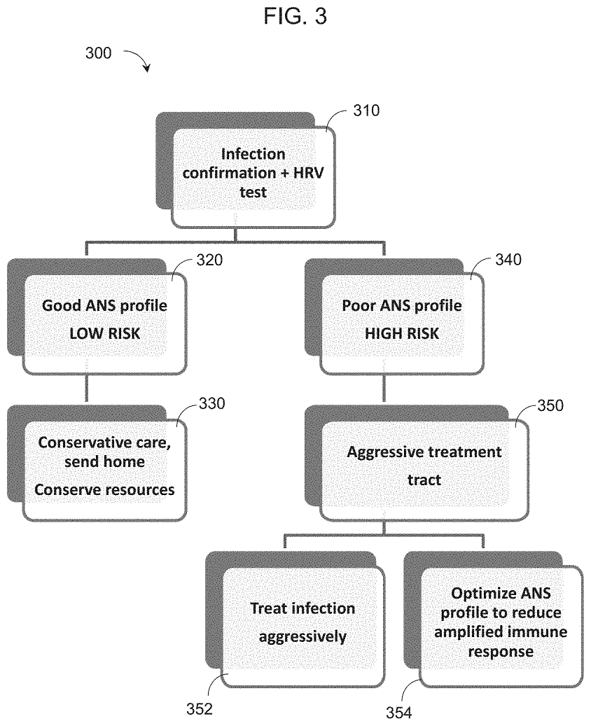

With reference to FIG. 3, an embodiment of a method of the present disclosure is described. In some embodiments, the method 300 includes confirming the subject has an infectious disease and performing an HRV test 310. In some embodiments, the HRV test includes measuring or obtaining a heartbeat pattern of the subject, and calculating an HRV based on the heartbeat pattern, as described herein. Upon determining the subject has a good ANS profile 320 based on the HRV test, the subject is deemed to have low risk of adverse disease progression (e.g., low risk of suffering a cytokine storm and/or ARDS). A conservative option for care or treatment may be selected 330 based on the good ANS profile. For example, the subject is given minimum treatment, or no treatment, or may not be hospitalized, thereby conserving medical resources. Upon determining the subject has a poor ANS profile 340 based on the HRV test, the subject is deemed to have high risk of adverse disease progression (e.g., high risk of suffering a cytokine storm and/or ARDS). An aggressive course or tract of treatment may be selected 350 based on the poor ANS profile. For example, the subject is administered 352 an aggressive treatment for the infectious disease. Alternatively, or in addition, the subject is treated to optimize 354 the subject's ANS profile (e.g., by administering an autonomous neuroregulatory therapy to reduce sympathetic activity and/or elevate parasympathetic activity), thereby controlling the subject's amplified immune response.

Also disclosed herein is a use of any form of heart rate variability (1996 Western or Russian) to stratify patient risk of death, hospitalization, and recovery from a pandemic infection, such as COVID-19. In some embodiments, ANS state, which can drive disease activity to cytokine storm, all other factors being equal, is measured in a subject.

In some embodiments, healthcare workers measure the ANS state of patient (e.g., using ANS Neuroscan™) while also screening for COVID-19. In some embodiments, if the patient is positive for infection, then the ANS state guides clinicians to stratify outcome and focus limited resources where they are most needed, e.g., for those with high sympathetic/low parasympathetic profile.

Also provided are methods of using any form of HRV to guide inclusion or exclusion of ANS optimizing therapeutics in a parallel tract with pandemic infectious disease treatments to improve overall outcomes (e.g., survival, hospitalization time, ICU time, cost of care, etc.).

Some embodiments of the present disclosure are further provided in the following numbered arrangements.

1. A method of assessing prognosis for an infectious disease, comprising:
measuring a heartbeat pattern of a subject having or suspected of having an infectious disease;
calculating a heart rate variability (HRV) based on the measured heartbeat pattern, wherein the HRV indicates sympathetic activity levels, parasympathetic activity levels and/or an autonomic nervous system (ANS) balance of the subject; and
assessing a prognosis for the infectious disease based on the HRV.

2. The method of arrangement 1, wherein assessing the prognosis comprises assessing a prognosis for a treatment for the infectious disease.

3. The method of arrangement 2, wherein the treatment for the infectious disease comprises treatment of the subject in an intensive care unit (ICU) and/or using ventilator support.

4. The method of any one of the preceding arrangements, wherein assessing the prognosis provides a favorable prognosis when the HRV indicates depressed sympathetic activity levels and/or elevated parasympathetic activity levels.

5. The method of any one of the preceding arrangements, wherein assessing the prognosis provides an unfavorable prognosis when the HRV indicates elevated sympathetic activity levels and/or depressed parasympathetic activity levels.

6. The method of any one of the preceding arrangements, further comprising treating the subject for the infectious disease based on the assessed prognosis.

7. The method of arrangement 6, comprising treating the subject for the infectious disease upon an assessment providing a favorable prognosis.

8. The method of arrangement 6, comprising treating the subject for the infectious disease upon an assessment providing an unfavorable prognosis.

9. The method of arrangement 6, comprising withholding a treatment for the infectious disease from the subject upon an assessment providing a favorable prognosis.

10. The method of arrangement 6, comprising withholding a treatment for the infectious disease from the subject upon an assessment providing an unfavorable prognosis.

11. The method of any one of the preceding arrangements, further comprising testing the subject for the infectious disease.

12. The method of arrangement 11, wherein testing is performed before measuring the HRV.

13. The method of any one of the preceding arrangements, further comprising selecting an ANS optimizing therapy based on the HRV, wherein the selected ANS optimizing therapy improves the prognosis for a treatment for the infectious disease when the ANS optimizing therapy is co-administered to the subject with the treatment.

14. The method of arrangement 13, wherein the ANS optimizing therapy comprises VNS and/or an ANS optimizing agent.

15. The method of arrangement 14, wherein the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist.

16. The method of arrangement 14, wherein the ANS optimizing agent comprises benzodiazepine or a selective serotonin reuptake inhibitor (SSRI).

17. The method of arrangement 14, wherein the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.

18. The method of arrangement 14, wherein the ANS optimizing agent comprises one or more of lorazepam, clonazepam, pramipexole or trazodone.

19. The method of any one of arrangements 13 to 18, further comprising selecting a treatment for the infectious disease.

20. The method of any one of arrangements 13 to 19, wherein the treatment comprises treatment of the subject in an intensive care unit (ICU), using a ventilator, and/or administration of a therapeutic agent to the subject.

21. A method of selecting an ANS optimizing therapy in a treatment for an infectious disease, comprising:
measuring a heartbeat pattern of a subject having an infectious disease;
calculating a heart rate variability (HRV) based on the measured heartbeat pattern, wherein the HRV indicates sympathetic activity levels, parasympathetic activity levels and/or an autonomic nervous system (ANS) balance of the subject; and
selecting an ANS optimizing therapy based on the HRV,
wherein the selected ANS optimizing therapy improves the prognosis for a treatment of the infectious disease when the ANS optimizing therapy is co-administered to the subject with the treatment.

22. The method of arrangement 21, comprising co-administering to the subject:
the treatment for the infectious disease; and
an effective amount of the selected ANS optimizing therapy.

23. The method of arrangement 21 or 22, comprising withholding an ANS optimizing therapy based on the measured HRV.

24. The method of any one of arrangements 21 to 23, wherein the treatment of the infectious disease comprises treatment of the subject in an intensive care unit (ICU) and/or using a ventilator.

25. The method of any one of arrangements 21 to 24, wherein the ANS optimizing therapy comprises vagus nerve stimulation (VNS) and/or an ANS optimizing agent.

26. The method of any one of arrangements 21 to 25, wherein the ANS optimizing therapy comprises VNS and/or an ANS optimizing agent.

27. The method of arrangement 26, wherein the ANS optimizing agent comprises an antidepressant, an anxiolytic, an anticonvulsant, a D₃ agonist or antagonist, a nicotinic acetylcholine receptor agonist or antagonist, an alpha-7 nicotinic receptor agonist or antagonist, a GABA_{A} receptor agonist or antagonist, an alpha-1 receptor agonist or antagonist, and an alpha-2 receptor agonist or antagonist.

28. The method of arrangement 27, wherein the ANS optimizing agent comprises benzodiazepine or a selective serotonin reuptake inhibitor (SSRI).

29. The method of arrangement 27, wherein the ANS optimizing agent comprises one or more of lorazepam, clonazepam, pramipexole, ropinirole, rotigotine, apomorphine, trazodone, pregabalin, imipramine, clomipramine, amitriptyline, maprotiline, fluvoxamine, paroxetine, fluoxetine, milnacipran, chlorodiazepoxide, diazepam, estazolam, oxazepam, bromazepam, alprazolam, midazolam, clobazam, clotiazepam, quazepam, clorazepate, flurazepam, triazolam, temazepam, etizolam, trans-N-{4-[4-(2,3-Dichlorophenyl)-1-piperazinyl]cyclohexyl}-3-methoxybenzamide, (−)-7-{[2-(4-Phenylpiperazin-1-yl)ethyl]propylamino}-5,6,7,8-tetrahydronaphthalen-2-ol, 5-OH-DPAT, 7-OH-DPAT, 8-OH-PBZI (cis-8-Hydroxy-3-(n-propyl)-1,2,3a,4,5,9b-hexahydro-1H-benz[e]indole), Apomorphine, Bromocriptine, Captodiame, CJ-1639, Dopamine, ES609, FAUC 54, FAUC 73, PD-128,907, PF-219,06, PF-592,379, Piribedil, Pramipexole, Quinelorane, Quinpirole, Ropinirole, Rotigotine, Amisulpride, Cyproheptadine, PG 01037, Domperidone, FAUC 365, GR-103,691, GSK598809, Haloperidol, N-(4-(4-(2,3-Dichloro- or 2-methoxyphenyl)piperazin-1-yl)butyl)heterobiarylcarboxamides, Nafadotride, NGB-2904, PNU-99,194, Raclopride, S-14,297, S33084, SB-277011-A, SR 21502, Sulpiride, U99194, YQA14, Bradanicline, Encenicline, Tropisetron, Anabasine, Acetylcholine, Nicotine, Epiboxidine, Ivermectin, Galantamine, Anandamide, α-Bungarotoxin, α-Conotoxin, Bupropion, Dehydronorketamine Ethanol, Hydroxybupropion, Hydroxynorketamine, Ketamine, Kynurenic acid, Memantine, Lobeline, Methyllycaconitine, Norketamine, Quinolizidine and gaboxadol, isoguvacine, muscimol, progabide, piperidine-4-sulfonic acid Risperidone.

30. The method of arrangement 27, wherein the ANS optimizing agent comprises one or more of lorazepam, clonazepam, pramipexole or trazodone.

31. The method of any one of arrangements 21 to 30, wherein the treatment comprises treatment of the subject in an intensive care unit (ICU), using a ventilator, and/or administration of a therapeutic agent to the subject.

32. The method of arrangement 19 or 31, wherein the therapeutic agent comprises an anti-infection agent, an immunosuppressant and/or an immunomodulator.

33. The method of arrangement 32, wherein the immunomodulator comprises one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

34. The method of arrangement 32, wherein the therapeutic agent comprises an anti-rheumatic drug.

35. The method of arrangement 32, wherein the therapeutic agent comprises an IL-6 inhibitor.

36. The method of arrangement 32, wherein the therapeutic agent comprises one or more of etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, ruxolitinib, tofacitinib, oclacitinib, baricitinib, filgotinib, cucurbitacin gandotinib, lestaurtinib, momelotinib, pacritinib, pf-04965842, upadacitinib, peficitinib.

37. The method of arrangement 32, wherein the anti-infection agent comprises one or more of an anti-viral agent, antibiotic, anti-fungal agent, and an anti-parasitic agent.

38. The method of arrangement 37, wherein the anti-viral agent comprises one or more of acyclovir, famcyclovir, sorivudine, trifluorothymidine, valacyclovir, dideoxyinosine, interferon alpha, lamivudine, rifampicin, baloxavir marboxil, famiciclovir, letermovir, Abacavir, Ziagen, Trizivir, Kivexa/Epzicom, Aciclovir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfinavir, Nevirapine, Nexavir, Nucleoside analogues, Novir, Oseltamivir (Tamiflu), Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Sofosbuvir, Stavudine, Tea tree oil, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir, and Zidovudine.

39. The method of arrangement 37, wherein the antibiotic comprises one or more of penicillin, amoxicillin, ampicillin, hetacillin, cloxacillin, dicloxacillin, methicillin, nafcillin, oxacillin, axlocillin, carbenicillin, mezlocillin, piperacillin, ticarcillin, cefadroxil, cefazolin, cephalixin, cephalothin, cephapirin, cephradine, cefaclor, cefacmandole, cefmetazole, cefonicid, ceforanide, cefotetan, cefoxitin, cefprozil, cefuroxime, loracarbef, cefixime, cefoperazone, cefotaxime, cefpodoxime, ceftazidime, ceftiofur, ceftizoxime, ceftriaxone, moxalactam, aztreonam, imipenem, eropenem, ciprofloxacin, enrofloxacin, difloxacin, orbifloxacin, marbofloxacin, chloramphenicol, thiamphenicol, florfenicol, chlortetracycline, tetracycline, oxytetracycline, doxycycline, minocycline, erythromycin, tylosin, tlimicosin, clarithromycin, azithromycin, lincomycin, clindamycin, gentamicin, amikacin, kanamycin, apramycin, tobramycin, neomycin, dihydrostreptomycin, paromomycin, sulfadmethoxine, sfulfamethazine, sulfaquinoxaline, sulfamerazine, sulfathiazole, sulfas alazine, sulfadiazine, sulfabromomethazine, suflaethoxypyridazine, vancomycin, teicoplanin, ramoplanin, decaplanin, rifampin, nitrofuran, virginiamycin, polymyxins, and tobramycin.

40. The method of arrangement 37, wherein the anti-fungal agent comprises one or more of itraconazole, ketoconazole, fluoconazole, voriconazole, griseofulvin and amphotericin B.

41. The method of any one of the preceding arrangements, wherein the infectious disease comprises a cold, pneumonia, or influenza.

42. The method of any one of the preceding arrangements, wherein the infectious disease comprises a viral infection, fungal infection, a bacterial infection, and/or a parasitic infection.

43. The method of any one of the preceding arrangements, wherein the infectious disease comprises an infection by one of more of a coronavirus, herpes simplex virus, papilloma virus, parainfluenza virus, influenza virus, hepatitis virus, Coxsackie Virus, herpes zoster virus, measles virus, mumps virus, rubella virus, rabies virus, hemorrhagic fever virus, and H1N1 virus.

44. The method of any one of the preceding arrangements, wherein the infectious disease comprises a coronavirus infection.

45. The method of arrangement 44, wherein the coronavirus infection comprises an infection by a Middle East respiratory syndrome coronavirus (MERS-CoV), severe acute respiratory syndrome coronavirus (SARS-CoV), or SARS-CoV-2 (COVID-19).

46. The method of any one of the preceding arrangements, wherein the infectious disease comprises an infection by one of more of Escherichia coli, Salmonella enterica, Shigella dysenteriae, Vibrio cholerae, Vibrio vulnificus, Vibrio parahaemolyticus, Virio vulnificus, Campylobacter jejuni, Klebsiella, Enterobacter, Serratia, Proteus, Providencia, and Morganella, Bacillus anthracis, Bacillus cereus, Clostridium tetani, Clostrium botulinum, Clostridium perfringens, Clostridium difficile, Mycobacterium tuberculosis, Legionella pneumophilla, Vibrio cholera, Staphylococcus aureus (such as Methicillin-resistant Staphylococcus aureus (MRSA)), Staphylococcus epidermidis, Staphylococcus saprophyticus, Streptococcus pyogenes, Streptococcus agalactiae, Enterococcus faecalis, Streptococcus bovis, Streptococcus pneumoniae, Streptococcus viridans, Pseudomonas aeruginosa, Corynebacterium diphtheriae, Listeria monocytogenes, Burcella, Francisella tularensis, Yersinia enterocolitica, Yersinia pseudotuberculosis, Yersinia pestis, Pasteurella multocida, Mycobacterium tuberculosis, Mycobacterium bovis, Mycobacterium avium, Mycobacterium leprae, Actinomyces israelii, Nocardia asteroides, Mycoplasma pneumoniae, Treponema pallidum, Borrelia brugdorferi, Leptospira interrogans, Chlamydia psittaci, Chlamydia trachomatis, Chlamydia pneumoniae, R. rickettsii, Coxiella burnetii, R. prowazekii, Gardnerella vaginalis, Lactobacillus, Peptococcus, Peptostreptococcus, Propionibacterium, Tropheryma, Burkholderia pseudomallei, and Burkholderia mallei.

47. The method of any one of the preceding arrangements, wherein the infectious disease is associated with acute respiratory distress syndrome (ARDS) and/or a cytokine storm.

48. The method of any one of the preceding arrangements, wherein a targeted treatment for the infectious disease is not available.

49. The method of any one of the preceding arrangements, wherein a vaccine effective for the infectious disease is not available.

50. The method of any one of the preceding arrangements, wherein the subject has acute respiratory distress syndrome (ARDS).

51. The method of any one of the preceding arrangements, wherein the subject has one or more comorbidities.

52. The method of arrangement 51, wherein the one or more comorbidities comprise hypertension, diabetes and coronary heart disease.

53. The method of any one of arrangements 1 to 50, wherein the subject does not have a comorbidity.

54. The method of any one of the preceding arrangements, wherein measuring the heartbeat pattern comprises electrocardiography or pulse wave telemetry.

55. The method of any one of the preceding arrangements, wherein the HRV comprises Western standard HRV or Russian HRV.

56. The method of any one of the preceding arrangements, wherein the HRV comprises time-domain HRV, geometric domain HRV, and/or frequency-domain HRV.

57. The method of arrangement 56, wherein the time-domain HRV comprises one or more of standard deviation between beats (SDNN), root mean square of successive differences (RMSSD), standard deviation of successive differences (SDSD), number of pairs of successive NNs that differ by more than 50 ms (NN50), proportion of NN50 divided by total number of NNs (pNN50), or estimated breath cycle (EBC).

58. The method of any one of the preceding arrangements, wherein the prognosis comprises one or more of chance of survival, ventilator time, ICU time, hospitalization time, time to recovery, rehabilitation time, back-to-employment time, permanent disability, near-term and/or extended-term mortality.

59. A computer system comprising a hardware processor and non-transitory memory comprising instructions stored thereon, wherein the instructions when executed by the hardware processor causes the processor to perform the method of any one of arrangements 1 to 5.

60. The computer system of arrangement 59, wherein the computer system comprises a tablet computer, a smartphone, a server or a desktop computer.

61. A computer-readable medium comprising a software program that comprises code for performing or causing performing the method of any one of arrangements 1 to 5.

62. The computer system of arrangement 59 or 60, or the computer-readable medium of arrangement 61, wherein the method further comprises selecting an ANS optimizing therapy based on the HRV, wherein the selected ANS optimizing therapy improves the prognosis for a treatment of the infectious disease when the ANS optimizing therapy is co-administered to the subject with the treatment.

63. The computer system or computer-readable medium of arrangement 62, wherein the ANS optimizing therapy comprises VNS and/or an ANS optimizing agent.

### EXAMPLES

### Example 1

A heartbeat pattern of a subject having an infectious disease (such as COVID-19) is measured. An HRV test indicative of parasympathetic activity level is calculated based on the measured heartbeat pattern. The calculated HRV parasympathetic output of 0.06 is low and indicates relatively poor parasympathetic activity, and provides an odds ratio of 9.0-25.0 [95% CI] of the subject needing a ventilation. Low parasympathetic state drives proinflammatory activity and higher risk of ARDS and death. Use of the ventilator is reserved for the subject. The subject is put on ventilator support if the subject's condition requires. An ANS optimization therapy is selected and administered to the subject to reduce the risk of the subject requiring ventilator support, since increasing parasympathetic state reduces proinflammatory activity and ARDS and death risk

### Example 2

A heartbeat pattern of a subject having an infectious disease (such as COVID-19) is measured. An HRV indicative of parasympathetic activity level is calculated based on the measured heartbeat pattern. The calculated parasympathetic output of 0.35 indicates a high parasympathetic activity, and provides an odds ratio of 11.3-22.9 [95% CI] of not requiring hospitalization at all. No treatment is selected for the subject, and the subject does not require hospitalization. No ANS optimization therapy is selected or administered to the subject, since favorable (high) parasympathetic state implies manageable, and not overactive and life-threatening, immune response to COVID-19 antigen.

### Example 3

A heartbeat pattern of a first subject having an infectious disease (such as COVID-19) is measured. The first subject is 80 years old with COPD. An HRV profile is calculated based on the measured heartbeat pattern.

A heartbeat pattern of a second subject having an infectious disease (such as COVID-19) is measured. The second subject is 50 years old with no comorbidities. An HRV profile is calculated based on the measured heartbeat pattern.

Combined, the HRV profile, age and COPD of the first subject indicates a poor prognosis and hospitalization is recommended to treat the first subject. Combined, the HRV profile, age and lack of comorbidities of the second subject indicates a favorable prognosis and no hospitalization is required for the second subject.

The above description discloses several methods and materials. This disclosed matter is susceptible to modifications in the methods and materials, as well as alterations in the fabrication methods and equipment. Such modifications will become apparent to those skilled in the art from a consideration of this disclosure or practice of the subject matter disclosed herein. Consequently, the specific embodiments disclosed herein are not intended to be limiting, and all modifications and alternatives coming within the true scope and spirit of the present disclosure are intended to be covered.

What is claimed is:

1. A method of assessing prognosis for an infectious disease, comprising:
measuring a heartbeat pattern of a subject having or suspected of having an infectious disease;
calculating a heart rate variability (HRV) based on the measured heartbeat pattern, wherein the HRV indicates sympathetic activity levels, parasympathetic activity levels and/or an autonomic nervous system (ANS) balance of the subject; and
assessing a prognosis for the infectious disease based on the HRV.
